(19) United States
(12) Patent Application Publication
(10) Pub. No.: US 2023/0027620 A1
(43) Pub. Date: Jan. 26, 2023

(54) PENTAPEPTIDE AND METHODS OF USE THEREOF

(71) Applicant: THE BOARD OF TRUSTEES OF THE UNIVERSITY OF ILLINOIS, Urbana, IL (US)

(72) Inventor: Vinay Kumar AAKALU, Chicago, IL (US)

(21) Appl. No.: 17/778,138

(22) PCT Filed: Nov. 25, 2020
(86) PCT No.: PCT/US2020/062140

§ 371 Date: May 19, 2022

Related U.S. Application Data
(60) Provisional application No. 62/941,226, filed on Nov. 27, 2019.

(51) Int. Cl. C07K 14/47; A61P 17/02; A61P 31/04; A61K 38/17; A61P 27/02; A61K 45/06

(52) U.S. Cl. CPC C07K 14/4723; A61P 17/02; A61P 31/04; A61K 38/1729; A61P 27/02; A61K 45/06

(57) ABSTRACT

A synthetic peptide including the sequence SHXGY (SEQ ID NO:2) is described as are methods of using the same for promoting wound healing and epithelial cell migration.

### INTRODUCTION

This application claims benefit of priority to U.S. Provisional Patent Application Ser. No. 62/941,226, filed Nov. 27, 2019, the content of which is incorporated herein by reference in its entirety.

This invention was made with government support under grant numbers EY024339 and EY029409 awarded by the National Institutes of Health; W81XWH-17-1-0122 awarded by the Department of Defense; and I01BX004080 awarded by the Department of Veterans Affairs Office of Research and Development. The government has certain rights in the invention.

### BACKGROUND

Histatins (HTNs) are small histidine-rich cationic peptides found in saliva, as well as human lacrimal epithelium (Aakalu, et al. (2014) Invest. Ophthalmol. Vis. Sci. 55:3115; Ubels, et al. (2012) Invest. Ophthalmol. Vis. Sci. 53(11):6738-47; Steele, et al. (2002) Invest. Ophthalmol. Vis. Sci. 43:98). Histatins range in size from 7 to 38 amino acid residues in length and represent a group of antimicrobial peptides with antibacterial properties and significant antifungal properties. In addition, histatins have been implicated in wound healing, metal ion chelation, anti-inflammatory effects and angiogenesis (Melino, et al. (2014) FEBS J. 281:657-72; Oudhoff, et al. (2008) FASEB J. 22(12):3805-12); Oudhoff, et al. (2009) J. Dent. Res. 88(9):846-50; WO 2007/142381). Structure-function studies have identified distinct N-terminal and C-terminal domains in both HTN1 and HTN3, which respectively contribute to the antimicrobial and wound healing properties (Melino, et al. (1999) Biochemistry 38:9626-33; Brewer, et al. (1998) Biochem. Cell Biol. 76:247-56; Gusman, et al. (2001) Biochim. Biophys. Acta 1545:86-95). In this respect, histatins, as well as fragments, multimers and combinations thereof, have been suggested for use in treating various conditions including ocular surface disease (US 2013/0310327; 2013/0310326; WO 2016/060916; WO 2016/060917; WO 2016/060918; WO 2016/060921; US 2016/0279194) and wounds (US 2013/0288964; US 2011/0178010).

Cyclic analogs of histatins have also been described. For example, U.S. Pat. No. 6,555,650 describes cyclic analogues of HTN5 with disulfide bridges that create a cyclic portion of from 5-16 of said amino acid units. In addition, head-to-tail cyclization of HTN5 has been shown to increase amphipathicity of the peptide without affecting its antimicrobial potency (Sikorska & Kamysz (2014) J. Pept. Sci. 20:952-7). Further, cyclization of histatin-1 has been shown to potentiate the molar activity approximately 1000-fold (Oudhoff, et al. (2009) FASEB J. 23:3928-35) and increases wound closure activity (Bolscher, et al. (2011) FASEB J. 25:2650-8). Moreover, cyclic analogs of histatin, with enhanced potency have been suggested for use in treating microbial infection (US 2010/0173833; Brewer & Lajoie (2002) Biochemistry 41:5526-5536).

### SUMMARY OF THE INVENTION

This invention provides a synthetic peptide, or a pharmaceutically acceptable salt thereof, where said peptide has the structure of Formula I:

Z—R¹-[L-R²]ₙ (I)

wherein at least one of R¹ or R² is a 5 to 10 amino acid residue peptide including the amino acid sequence SHXGY (SEQ ID NO:1), wherein X is R, K, H, D or E and the other of R¹ or R² is a metal binding peptide, wound healing peptide, or antimicrobial peptide; Z is present or absent and when present is an exogenous peptide; L is a linker; and n is 0 or ≥1, with the proviso that when n is 0, R¹ is a 5 to 10 amino acid residue peptide comprising the amino acid sequence SHXGY (SEQ ID NO:1). In certain aspects, each occurrence of L may include the same or different linker; the synthetic peptide may be linear or cyclized; and/or the peptide may include a modification selected from glycosylation, acetylation, amidation, formylation, hydroxylation, methylation, myristoylation, phosphorylation, sulfonation, PEGylation or lipidation. In other aspects, the metal binding peptide may have the amino acid sequence HEXXH (SEQ ID NO:14), wherein X is K, R, or H; the wound healing peptide may have the amino acid sequence SNYLYDN (SEQ ID NO:26) or SHXGY (SEQ ID NO:1), wherein X is R, K, H, D or E; and the antimicrobial peptide may have the amino acid sequence RKFHEKHHSHRGYR (SEQ ID NO:28) or AKRHHGYKRKFH (SEQ ID NO:29). A pharmaceutical composition including one or more of the synthetic peptides, or pharmaceutically acceptable salt thereof, in admixture with a pharmaceutically acceptable carrier or excipient is also provided, as is a kit and methods for promoting wound healing and/or epithelial cell migration, and increasing extracellular signal-regulated protein kinase (ERK) activation using the synthetic peptide, or pharmaceutically acceptable salt thereof, or a pharmaceutical composition containing the same, wherein the amount of synthetic peptide, or pharmaceutically acceptable salt thereof, administered is in the range of 1 nanomolar to 500 micromolar. In some aspects, the kit and method may further include the use of an antimicrobial agent, an antiviral agent, an antiparasitic agent, an immunomodulatory agent, an anti-scarring agent, collagen, gelatin, a pain reliever, an anesthetic agent, or a combination thereof. Furthermore, pentapeptides prepared in accordance with the principles of the present disclosure can be manipulated to be multiplied, oriented in linear arrangement or other configurations and maintain their function.

### BRIEF DESCRIPTION OF THE DRAWINGS

FIG. 1 shows that SHRGY (SEQ ID NO:2)-containing peptides are necessary and sufficient for acceleration of scratch closure rates. Peptides were tested for efficacy in accelerating scratch closure rates. Negative controls included untreated and multiple scrambled peptide (SP) controls. All peptides were tested at 80 μM concentration. All experiments were performed in triplicate with three technical replicates for each experiment. Statistical significance was determined by one-way ANOYA with Dunnett's post-hoc test. *p<0.05; **p<0.01; ***p<0.001. Relative closure=(% closure of treated sample)/(% closure of Untreated control).

FIG. 2 shows that application of Histatin-5 or SHRGY (SEQ ID NO:2) peptides accelerates corneal wound closure rates in a murine corneal epithelial injury model. Wounded areas were treated with Hst5, SHRGY (SEQ ID NO:2) or SP1 peptides (n=7 for each group), each tested at 80 μM concentration. Wound areas at multiple time points were measured using Image J software. Measurement of % remaining corneal wound area at 18 hours and 24 hours compared to baseline showed statistically significant improvement in Hst5 and SHRGY (SEQ ID NO:2) groups compared to SP1. Statistical significance was determined by a two-way ANOVA with Bonferroni post-hoc test. **P<0.01, ***P<0.001. % Wound area=(Wound area at time X/Wound area at time 0)×100.

FIG. 3 shows scratch closure rates for different salt forms of the SHRGY (SEQ ID NO:2) peptide. Human corneal epithelial cells were scratched using standardized scratch assays and tested for increased rates of wound healing after application of different salt forms of pentapeptide. AA, acetic acid; HCL, hydrochloride; TFA, trifluoroacetate.

### DETAILED DESCRIPTION OF THE INVENTION

Epithelial migration, adhesion and proliferation are critical to wound healing in all areas of the body. Without adequate epithelialization, aberrant wound healing, with consequences such as scarring, inflammation, pain, contractures, visual loss, functional compromise, infection and abnormal blood vessel proliferation can be seen. Several cell types and wounding models are commonly used to test the applicability of agents to enhance wound healing. An exemplary system is the anterior segment and ocular surface of the eye. The cornea and other elements of the anterior surface of the eye are used as models for wound healing as aberrant epithelialization can lead to devastating loss of vision or even loss of the eye. It has now been found that the pentapeptide SHXGY (SEQ ID NO:1) is capable of enhancing epithelial wound healing. In particular, it has been shown that peptides containing the sequence SHXGY (SEQ ID NO:1), e.g., SHRGY (SEQ ID NO:2) and SHDGY (SEQ ID NO:3), can significantly enhance epithelial migration, in several cell types (immortalized human corneal epithelial cells, immortalized human corneal limbal epithelial cells, HeLa human cells) and in models of mouse corneal epithelial wounding. When applied topically to mice, which have been injured using a standardized wounding method, SHXGY (SEQ ID NO:1)-containing peptides or multimers thereof (e.g., SHRGY-(CH₂)₆-SHRGY-(CH₂)₆-SHRGY-(CH₂)₆-SHRGY; SEQ ID NO:51) can significantly improve corneal healing. In addition, synthetic peptides including the SHRGY (SEQ ID NO:2) sequence were shown to stimulate immunolocalization of phosphorylated extracellular signal-regulated protein kinases 1/2 (pERK1/2) to the site of wound healing, thereby suggesting that, in addition to its function in enhancing would healing, the SHRGY (SEQ ID NO:2) peptide also has immunomodulatory activity. Thus, this core pentapeptide sequence is of particular use in promoting epithelialization and cell migration, which is important to wound healing, inflammation, cancer, responses to infection or injury amongst other phenomena.

Accordingly, this invention provides a synthetic peptide, or a pharmaceutically acceptable salt thereof, and methods of use of the same in promoting wound healing and/or epithelial cell migration. A synthetic peptide of this invention has the general structure of Formula I:

Z—R¹-[L-R²]ₙ (I),

wherein

(i) at least one of R¹ or R² is a 5 to 10 amino acid residue peptide having the amino acid sequence SHXGY (SEQ ID NO:1), wherein X is R, K, H, D or E and the other of R¹ or R² is a metal binding peptide, wound healing peptide, or antimicrobial peptide;

(ii) Z is present or absent and when present is an exogenous peptide;

(iii) L is a linker; and

(iv) n is 0 or ≥1

with the proviso that when n is 0, R¹ is a 5 to 10 amino acid residue peptide having the amino acid sequence SHXGY (SEQ ID NO:1).

As indicated, at least one of R¹ and R² is a 5 to 10 amino acid residue peptide that includes the amino acid sequence SHXGY (SEQ ID NO:1), wherein X is R (Arg), K (Lys), H (His), D (Asp) or E (Glu). Accordingly, at least one of R¹ and R² is a 5, 6, 7, 8, 9 or 10 amino acid residue peptide that includes the amino acid sequence SHRGY (SEQ ID NO:2), SHDGY (SEQ ID NO:3), SHKGY (SEQ ID NO:4), SHHGY (SEQ ID NO:5), or SHEGY (SEQ ID NO: At least one of R¹ or R² includes the sequence SHXGY (SEQ ID NO:1), which may have 1 to 5 additional amino acid residues on the C-terminus and/or N-terminus. In some aspects, the 1 to 5 additional amino acid residues are endogenous or native amino acid residues. A “native” or “endogenous” amino acid residue is an amino acid residue that is present at the recited position in a naturally occurring protein. By way of illustration, the sequence SHRGY (SEQ ID NO:2) is present within histatin 3 as follows:

(SEQ ID NO: 7)
DSHAKRHHGYKRKFHEKHHSHRGYRSNYLYDN.

Accordingly, when R¹ and/or R² is derived from a histatin, R¹ and/or R² can have the sequence HHSHRGYRSN (SEQ ID NO:8), HEKHHSHRGY (SEQ ID NO:9), EKHHSHRGYR (SEQ ID NO:10), KHHSHRGY (SEQ ID NO:11), HHSHRGY (SEQ ID NO:12), or HSHRGY (SEQ ID NO:13).

In some aspects, the synthetic peptide consists only of R¹ (i.e., n=0). In accordance with this aspect, the synthetic peptide is a 5, 6, 7, 8, 9 or 10 amino acid residue peptide comprising or consisting of the sequence set forth in SEQ ID NO:2, SEQ ID NO:3, SEQ ID NO:4, SEQ ID NO:5, or SEQ ID NO:6.

In other aspects, the synthetic peptide includes one or more R² peptides (i.e., n≥1). In this respect, the synthetic peptide can include 2, 3, 4, 5, 6, 7, 8, 9, 10, 11, 12, 13, 14, 15, 16, 17, 18, 19, 20 or more peptides joined by linkers. In one aspect R¹ and R² of the synthetic peptide of this invention are the same. In other aspects, R¹ and R² of the synthetic peptide of this invention are different. In further aspects, each R² can be the same or different. Ideally, the total length the synthetic peptide is in the range of 20 to 100 amino acid residues.

While at least one of R¹ or R² is a 5 to 10 amino acid residue peptide having the amino acid sequence SHXGY (SEQ ID NO:1), the other of R¹ or R² may be a metal binding peptide, wound healing peptide, or antimicrobial peptide. In this respect, a synthetic peptide of the invention may be composed of a 5 to 10 amino acid residue peptide having the amino acid sequence SHXGY in combination with (i) a metal binding peptide, (ii) a wound healing peptide, (iii) an antimicrobial peptide, or (iv) any combination of (i)-(iii). In certain aspects, a synthetic peptide of the invention is composed of a 5 to 10 amino acid residue peptide having the amino acid sequence SHXGY in combination with a second wound healing peptide.

The term “metal binding peptide,” as used herein, refers to an amino acid motif that binds or forms a complex with a metal. Structural and functional characterization of histatins has revealed the presence of two metal-binding motifs: the amino-terminal Cu(II)/Ni(II) binding (ATCUN) motif with one histidine residue in the third position (NH₂—X₁X₂H, wherein X₁ is Asp or Glu, and X₂ is Ala, Thr, Met or Ser) (Grogan, et al. (2001) FEBS Lett. 491:76-80; Melino, et al. (2006) Biochemistry 45:15373-83; Melino, et al. (1999) Biochemistry 38:9626-33; Gusman, et al. (2001) Biochim. Biophys. Acta 1545:86-95); and the Zn(II)-binding motif HEXXH (SEQ ID NO:14), wherein X denotes a basic amino acid residue such as K (Lys), R (Arg), or H (His). Accordingly, in some embodiments, the metal binding peptide includes the sequence DSH, ESH, DAH, EAH, DTH, ETH, DMH or EMH. In other embodiments, the metal binding peptide includes the sequence HEKKH (SEQ ID NO:15), HEKRH (SEQ ID NO:16), HEKHH (SEQ ID NO:17), HERKH (SEQ ID NO:18), HERRH (SEQ ID NO:19), HERHH (SEQ ID NO:20), HEHKH (SEQ ID NO:21), HEHRH (SEQ ID NO:22) or HEHHH (SEQ ID NO:23). The metal binding peptide can include the specific sequence of the above-referenced metal binding peptides or can include between 1 and 6 additional native histatin amino acid residues on the C- and/or N-terminus of the metal binding peptide. By way of illustration, a metal binding peptide can have the sequence GYKRKFHEKHHSHR (SEQ ID NO:24) or HEKRHH (SEQ ID NO:25).

In some embodiments, a synthetic peptide of the invention includes one metal binding peptides. In other embodiments, a synthetic peptide includes two metal binding peptides. In further embodiments, a synthetic peptide includes three metal binding peptides. In certain embodiments, a metal binding peptide has the sequence HEXXH (SEQ ID NO:14), wherein each X is a basic amino acid residue. As would be readily appreciated by those of skill in the art, the inclusion of one or more metal binding peptides in a synthetic peptide impart metal ion chelating, anti-inflammatory, matrix metalloproteinase inhibitory, and/or anti-angiogenic activity to the synthetic peptide. In light of its anti-angiogenic activity, such a synthetic peptide would be of use in treating age-related macular degeneration, diabetic retinopathy, cancer, and chronic or acute sever uveitis. In light of its metal ion chelating activity, such a synthetic peptide would also be of use in inhibiting tissue destruction mediated by matrix metalloproteinases and other metal-dependent enzymes in inflammatory and infectious diseases such as infectious keratitis, intraocular uveitis, endophthalmitis, inflammatory keratitis, dry eye disease and ocular surface or intraocular diseases.

As used herein, “wound healing peptide” refers to an amino acid motif that promotes or facilitates wound healing. In some aspects, a wound healing peptide is derived from histatin. An example of a wound healing peptide derived from histatin is a peptide including the sequence SNYLYDN (SEQ ID NO:26). In another aspect, the wound healing peptide includes the amino acid sequence SHXGY (SEQ ID NO:1), wherein X is R, K, H, D or E. Notably, when included in the synthetic peptide of this invention, the SHXGY (SEQ ID NO:1) sequence has the additional advantage of conferring immunomodulatory activity to the synthetic peptide. The wound healing peptide can include the specific sequence of the above-referenced wound healing peptides or can include between 1 and 6 additional amino acid residues on the C- and/or N-terminus of the wound healing peptide. By way of illustration, a wound healing peptide derived from histatin can have the sequence YGDYGSNYLYDN (SEQ ID NO:27) or any one of SEQ ID NO:2 or 8-13.

In some embodiments, in addition to the wound healing peptide of SEQ ID NO:1, the synthetic peptide of the invention includes a second wound healing peptide. In other embodiments, in addition to the wound healing peptide of SEQ ID NO:1, a synthetic peptide includes two additional wound healing peptides. In further embodiments, in addition to the wound healing peptide of SEQ ID NO:1, a synthetic peptide includes three additional wound healing peptides. As would be readily appreciated by those of skill in the art, the inclusion of one or more wound healing peptides in a synthetic peptide impart epithelial cell migration and spreading activity to the synthetic peptide. Such a synthetic peptide would therefore be of use in wound healing as well as the treatment of retinal pigment epithelial healing, dry age-related macular degeneration, ocular surface diseases and ocular surface inflammatory disorders, ocular neovascularization including corneal and intraocular, retinal or choroidal, and dry eye diseases.

For the purposes of this invention, “antimicrobial” includes both antibacterial and antifungal agents. Accordingly, the term “antimicrobial” peptide,” as used herein, refers to an amino acid motif that exhibits cytostatic or cytocidal activity toward bacterial and/or fungal cells. Characterization of histatins indicates that a positive net charge and the amino-terminal portion of HTNs mediate antimicrobial activity. In particular, the amino acid sequence RKFHEKHHSHRGYR (SEQ ID NO:28) of HTN3 has been shown to exhibit fungicidal activity (Oppenheim, et al. (2012) PLoS ONE 7(12):e51479). Similarly, the sequence AKRHHGYKRKFH (SEQ ID NO:29), also known as P-113, exhibits fungicidal activity against Candida albicans (Jang, et al. (2008) Antimicrob. Agents Chemother. 5292):497-504). Thus, the antimicrobial peptide can include the specific sequence of the above-referenced antimicrobial peptides or can include between 1 and 6 additional amino acid residues on the C- and/or N-terminus of the antimicrobial peptide.

In some embodiments, a synthetic peptide includes one antimicrobial peptide. In other embodiments, a synthetic peptide includes two antimicrobial peptides. In further embodiments, a synthetic peptide includes three antimicrobial peptides. In certain embodiments, an antimicrobial peptide has the sequence RKFHEKHHSHRGYR (SEQ ID NO:28). In other embodiments, an antimicrobial domain has the sequence AKRHHGYKRKFH (SEQ ID NO:29). As would be readily appreciated by those of skill in the art, the inclusion of one or more antimicrobial peptides in a synthetic peptide impart antifungal and/or antibacterial activity to the synthetic peptide. Such a synthetic peptide would therefore be of use in treating microbial infections such as Candida eye infection as well as preventing infections associated with surgical implants.

Examples of synthetic peptides containing repeating units that are the same or different are presented in Table 1.

TABLE 1
Synthetic Peptide

SHRGY (SEQ ID NO: 2)-L-SHRGY (SEQ ID NO: 2)-L-SHRGY (SEQ ID NO: 2)-
L-SHRGY (SEQ ID NO: 2)
SHRGY (SEQ ID NO: 2)-L-SHDGY (SEQ ID NO: 3)-L-SHRGY (SEQ ID NO: 2)-
L-SHDGY (SEQ ID NO: 3)
SHRGY (SEQ ID NO: 2)-L-SHRGY (SEQ ID NO: 2)-L-SHDGY (SEQ ID NO: 3)-
L-SHDGY (SEQ ID NO: 3)-L-SHRGY (SEQ ID NO: 2)-L-SHRGY (SEQ ID NO: 2)
HHSHRGY (SEQ ID NO: 12)-L-SHRGY (SEQ ID NO: 2)-L-HSHRGY (SEQ ID NO: 13)-
L-SHDGY (SEQ ID NO: 3)-L-SHRGY (SEQ ID NO: 2)
HEKHH (SEQ ID NO: 17)-L-SHRGY (SEQ ID NO: 2)
HEKHH (SEQ ID NO: 17)-L-HEKHH (SEQ ID NO: 17)-L-SHRGY (SEQ ID NO: 2)-
L-YGDYGSNYLYDN (SEQ ID NO: 27)
SHRGY (SEQ ID NO: 2)-L-HEKRHH (SEQ ID NO: 25)-L-HEKRHH (SEQ ID NO: 25)-
L-YGDYGSNYLYDN (SEQ ID NO: 27)

In certain aspects of this invention, exogenous or heterologous molecules are included in the synthetic peptide. Specifically, in some aspects, the synthetic peptide includes “Z” and/or “L” moieties directly attached to one or both of R¹ and R², wherein both “Z” and “L” moieties are exogenous or heterologous molecules with respect to R¹ and R². The term “heterologous molecule” or “exogenous molecule” refers to a molecule that is not normally found in a peptide or not typically associated with R¹ and/or R² amino acid sequences in nature.

In some aspects, the synthetic peptide includes a “Z” moiety. In other aspects, the “Z” moiety is absent. When present, Z is an exogenous peptide as defined herein. In accordance with this aspect, Z is a 1 to 50 amino acid residue peptide, or preferably a 1 to 30 amino acid residue peptide, or more preferably a 1 to 20 amino acid residue peptide, wherein said exogenous peptide may or may not have a function. By way of illustration, the exogenous peptide may exhibit metal binding, wound healing, immunomodulatory, and/or antimicrobial activity or may be a random peptide sequence, e.g., as in the SP2 peptide HSHKEGHHYKRFKRKHHADSHRGY (SEQ ID NO:70). In certain aspects, Z is a 1-50, 1-30 or 1-20 amino acid residue random peptide sequence.

As used herein, the terms “L” or “linker” or “spacer” refers to a heterologous or exogenous molecule used to connect, link or join R¹ to R² and connect, link or join individual R² moieties. As used herein, the term “linked,” “joined” or “connected” generally refers to a functional linkage between two contiguous or adjacent amino acid sequences to produce a molecule that does not exist in nature. Generally, the linked amino acid sequences are contiguous or adjacent to one another and retain their respective operability and function when joined. The linkers may provide desirable flexibility to permit the desired expression, activity and/or conformational positioning of the synthetic peptide.

In some embodiments, a synthetic peptide includes one linker, i.e., n=1. In other embodiments, a synthetic peptide includes 2, 3, 4, 5, 6, 7, 8, 9, 10, 11, 12, 13, 14, 15, 16, 17, 18, or 19 linkers, i.e., n=2, 3, 4, 5, 6, 7, 8, 9, 10, 11, 12, 13, 14, 15, 16, 17, 18, or 19. In certain aspects, each occurrence of a linker (L) may include the same or different linker.

Linkers of use in the synthetic peptide of Formula I can be flexible, rigid, in vivo cleavable, or a combination thereof. In addition, linkers can be composed of amino acid residues (i.e., peptide linkers) or composed of chains of hydrocarbons (i.e., hydrocarbon linkers). Peptide linkers can be of any appropriate length to connect R¹ and R² or individual R² moieties and are preferably designed so as to allow the proper folding and/or function and/or activity of R¹ and R². Thus, the linker peptide can have a length of no more than 3, no more than 5, no more than 10, no more than 15, no more than 20, no more than 25, no more than 30, no more than 35, no more than 40, no more than 45, no more than 50, no more than 55, or no more than 60 amino acids. In some embodiments, the linker peptide can have a length of at least 3, at least 4, at least 5, at least 6, at least 7, at least 8, at least 9, at least 10, at least 12, at least 15, at least 18, at least 20, at least 25, at least 30, at least 35, at least 40, at least 45, or at least 50 amino acids. In some embodiments, the linker includes at least 10 and no more than 60 amino acids, at least 10 and no more than 55 amino acids, at least 10 and no more than amino acids, at least 10 and no more than 45 amino acids, at least 10 and no more than 40 amino acids, at least 10 and no more 35 amino acids, at least 10 and no more than 30 amino acids, at least 10 and no more than 25 amino acids, at least 10 and no more than 20 amino acids or at least 10 and no more than 15 amino acids.

A “flexible” linker refers to a hydrocarbon or peptide linker that does not have a fixed structure (secondary or tertiary structure) in solution. Such a flexible linker is therefore free to adopt a variety of conformations. Flexible linkers of use herein include hydrocarbon linkers and peptide linkers composed of small, non-polar (e.g., Gly) and/or polar (e.g., Ser or Thr) amino acid residues. Simple amino acids (e.g., amino acids with simple side chains (e.g., H, CH₃ or CH₂OH) are advantageous for use in a peptide linker as the lack of branched side chains on these amino acids provides greater flexibility (e.g., two-dimensional or three-dimensional flexibility) within the linker and, accordingly, within a polypeptide composition. The flexible linker may contain additional amino acids such as Thr and Ala to maintain flexibility, as well as polar amino acids such as Lys and Glu to improve solubility. The amino acids can alternate/repeat in any manner consistent with the linker remaining functional (e.g., resulting in expressed and/or active polypeptide(s)). Flexible linkers are described, for example, in Chen, et al. (2013) Adv. Drug Deliv. Rev. 65(10):1357-1369; US 2012/0232021; US 2014/0079701; WO 1999/045132; WO 1994/012520 and WO 2001/1053480.

In particular aspects, the flexible linker is a hydrocarbon linker. The hydrocarbon linking R¹ and R² or individual R² moieties should have sufficient length and flexibility so that the synthetic peptide can achieve the desired conformation. In certain embodiments, the hydrocarbon is composed of one or more methylene (—CH₂—) groups. In certain embodiments, the hydrocarbon includes between 3 and 25 methylene groups, i.e., —(CH₂)ₙ—, wherein n is 3 to 25. In certain embodiments, the hydrocarbon linker has the structure —(CH₂)₆—. Additional carbon-based linkers such as glycol linkers could also be used in the synthetic peptide of this invention.

In other embodiments, the linker is a rigid linker. “Rigid” linker refers to a molecule that adopts a relatively well-defined conformation when in solution. Rigid linkers are therefore those which have a particular secondary and/or tertiary structure in solution. Rigid linkers are typically of a size sufficient to confer secondary or tertiary structure to the linker. Such linkers include aromatic molecules (see, e.g., U.S. Pat. No. 6,096,875 or U.S. Pat. No. 5,948,648), peptide linkers rich in proline, or peptide linkers having an inflexible helical structure. Rigid linkers are described in, for example, Chen, et al. (2013) Adv. Drug Deliv. Rev. 65(10):1357-1369; US 2010/0158823 and US 2009/10221477.

In other embodiments, the linker is an in vivo cleavable linker. In vivo cleavable linkers can include a cleavable disulfide bond formed between two cysteine residues or linkers having a protease recognition sequence, e.g., recognized by matrix metalloproteases (MMPs).

Examples of suitable peptide linkers of use in the synthetic peptide are provided in Table 2.

TABLE 2

| Type | Sequence | SEQ ID NO: |
|---|---|---|
| Flexible | (GGGGS)ₙ | 30 |
| Flexible | KESGSVSSEQLAQFRSLD | 31 |
| Flexible | EGKSSGSGSESKST | 32 |
| Flexible | GGGGGGGG | 33 |
| Flexible | GSAGSAAGSGEF | 34 |
| Flexible | (GGSG)ₙ | 35 |
| Flexible | (GS)ₙ | 36 |
| Rigid | (EAAAK)ₙ | 37 |
| Rigid | A(EAAAK)ₙA | 38 |
| Rigid | PAPAP | 39 |
| Rigid | (XP)ₙ | 40 |
| Cleavable | VSQTSKLTRAETVFPDV | 41 |
| Cleavable | PLGLWA | 42 |
| Cleavable | RVLAEA | 43 |
| Cleavable | EDVVCCSMSY | 44 |
| Cleavable | GGIEGRGS | 45 |
| Cleavable | TRHRQPRGWE | 46 |
| Cleavable | AGNRVRRSVG | 47 |
| Cleavable | RRRRRRRRR | 48 |
| Cleavable | GFLG | 49 |
| Cleavable | CRRRRRREAEAC | 50 |

n is 1 to 5. X may be any amino acid residue, but is preferably Ala, Lys or Glu.

Each of the individual linkers of the synthetic peptide of this invention can be the same or different. In some embodiments, a synthetic peptide includes at least one flexible linker. In some embodiments, at least one flexible linker is a hydrocarbon linker. In other embodiments, at least one flexible linker is a peptide linker. In particular embodiments, each linker of the synthetic peptide is a hydrocarbon linker. In certain embodiments, each linker of the synthetic peptide has the structure —(CH₂)₆—.

Examples of synthetic peptides containing combinations repeating units with flexible linkers are presented in Table 3.

TABLE 3

| Synthetic Histatin | SEQ ID NO: |
|---|---|
| SHRGY-(CH₂)₆-SHRGY-(CH₂)₆-SHRGY-(CH₂)₆-SHRGY | 51 |
| SHRGY-(CH₂)₆-SHDGY-(CH₂)₆-SHRGY-(CH₂)₆-SHDGY | 52 |
| SHRGY-GGGGGGGG-SHRGY-GGGGS-SHRGY-GGGGGGGG-SHRGY | 53 |
| SHRGY-GGGGS-SHRGY-GGGGS-SHRGY-GGGGS-SHRGY | 54 |
| SHRGY-GGGGGGGG-SHRGY-PAPAP-SHRGY-GGGGGGGG-SHRGY | 55 |

In some aspects, a synthetic peptide of the invention is prepared as a pharmaceutically acceptable salt. As used herein, the term “pharmaceutically acceptable salt” refers to those salts of the synthetic peptide which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well-known in the art. See, e.g., Berge, et al. (1977) J. Pharmaceutical Sciences 66:1-19. Salts can be prepared in situ during the final isolation and purification of the peptides of the invention, or separately by reacting a free base with a suitable organic acid. Examples of pharmaceutically acceptable salts include, but are not limited to, nontoxic acid addition salts formed from amino group and an inorganic acids such as hydrochloric acid, hydrobromic acid, phosphoric acid, sulfuric acid and perchloric acid or with organic acids such as acetic acid, maleic acid, tartaric acid, citric acid, succinic acid or malonic acid or by using other methods used in the art such as ion exchange. Other pharmaceutically acceptable salts include, but are not limited to, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, alkyl having from 1 to 6 carbon atoms, sulfonate and aryl sulfonate.

The synthetic peptides described herein are commonly referred to as fusion or chimeric peptides. Such molecules can be synthesized by routine methods including recombinant protein expression, chemical synthesis, or a combination thereof. In some embodiments, the synthetic peptide of the invention is synthesized recombinantly using recombinant DNA techniques. Thus, the invention provides polynucleotides that encode the synthetic peptide of the invention. In a related aspect, the invention provides vectors, particularly expression vectors that harbor the polynucleotides encoding the synthetic peptide of the invention. In certain embodiments, the vector provides replication, transcription and/or translation regulatory sequences that facilitate recombinant synthesis of the desired synthetic histatin in a eukaryotic cell or prokaryotic cell. Accordingly, the invention also provides host cells for recombinant expression of the synthetic peptide and methods of harvesting and purifying the synthetic peptide produced by the host cells. Production and purification of recombinant peptides is a routine practice to one of skilled in the art and any suitable methodology can be used.

In another embodiment, the synthetic peptide is synthesized by any of the chemical synthesis techniques known in the art, particularly solid-phase synthesis techniques, for example, using commercially-available automated peptide synthesizers. See, for example, Stewart & Young (1984) Solid Phase Peptide Synthesis, 2^{nd} ed., Pierce Chemical Co.; Tarn, et al. (1983) J. Am. Chem. Soc. 105:6442-55; Merrifield (1986) Science 232:341-347; and Barany et al. (1987) Int. J. Peptide Protein Res. 30:705-739.

The synthetic peptide can be isolated and/or purified by any suitable methods known in the art including without limitation gel filtration and affinity purification. In some embodiments, the synthetic peptide is produced with a tag, e.g., an epitope tag, to facilitate isolation of the synthetic peptide. In one aspect, the synthetic peptide is at least 1% pure, e.g., at least 5% pure, at least 10% pure, at least 20% pure, at least 40% pure, at least 60% pure, at least 80% pure, and at least 90% pure, as determined by SDS-PAGE. Once isolated and/or purified, the properties of the synthetic peptide can be readily verified by techniques known to those skilled in the art.

Derivatives and analogs of the synthetic peptide described herein are all contemplated and can be made by altering their amino acid sequences by substitutions, additions, and/or deletions/truncations or by introducing chemical modifications that result in functionally equivalent molecules. It will be understood by one of ordinary skill in the art that certain amino acids in a sequence of any polypeptide may be substituted for other amino acids without adversely affecting the activity of the polypeptides.

In certain embodiments, the synthetic peptide of the invention includes one or more modifications including without limitation phosphorylation, glycosylation, hydroxylation, sulfonation, amidation, acetylation, carboxylation, palmitylation, PEGylation, introduction of nonhydrolyzable bonds, and disulfide formation. The modification may improve the stability and/or activity of the synthetic peptide.

For example, the C-terminal may be modified with amidation, addition of peptide alcohols and aldehydes, addition of esters, or addition of p-nitroaniline and thioesters. The N-terminal and side chains may be modified by PEGylation, acetylation, formylation, addition of a fatty acid, addition of benzoyl, addition of bromoacetyl, addition of pyroglutamyl, succinylation, addition of tetrabutyoxycarbonyl and addition of 3-mercaptopropyl, acylations (e.g., lipopeptides), biotinylation, phosphorylation, sulfation, glycosylation, introduction of maleimido group, chelating moieties, chromophores or fluorophores.

In one embodiment, the synthetic peptide is conjugated to a fatty acid, e.g., the synthetic peptide is myristoylated. For example, a fatty acid may be conjugated to the N-terminus of the synthetic peptide. Such fatty acids include caprylic acid, capric acid, lauric acid, myristic acid, palmitic acid, stearic acid, etc. Furthermore, cysteines in synthetic peptide can be palmitoylated. In one embodiment, the synthetic peptide is myristylated, stearylated or palmitoylated at the N-terminal amino acid.

In addition, or as an alternative, to post-translational modifications, the synthetic peptide can be conjugated or linked to another peptide, such as a carrier peptide. The carrier peptide may facilitate cell-penetration and can include peptides such as antennapedia peptide, penetratin peptide, TAT, transportan or polyarginine. In an embodiment, the synthetic peptide is conjugated or linked to the antennapedia peptide, RQIKIWFQNRRMKWKK (SEQ ID NO:56).

A synthetic peptide of the invention may also be cyclized. As used herein the term “cyclized” or “cyclic” denote an analog of a linear peptide that incorporates at least one bridging group (e.g., an amide, thioether, thioester, disulfide, urea, carbamate, hydrocarbon or sulfonamide) between to amino acid residues to form a cyclic structure. The bridging group can present on the side chain of an amino acid residue or a terminal amino acid residue thereby providing side chain cyclization (e.g., lactam bridge, thioester), head-to-tail cyclization, or hydrocarbon-stapled peptides.

In certain embodiments, the cyclic synthetic peptide has a disulfide bridge between two terminal cysteine residues. Representative amino acid sequences for preparing cyclized synthetic peptides are provided in Table 4.

TABLE 4

| | SEQ ID |
|---|---|
| Cyclic Synthetic Peptide | NO: |
| C-SHRGY-(CH2)₆-SHRGY-(CH₂)₆-SHRGY-(CH₂)₆-SHRGY-C | 57 |
| C-SHRGY-(CH2)₆-SHDGY-(CH₂)₆-SHRGY-(CH₂)₆-shdgy-C | 58 |
| C-SHRGY-GGGGGGGG-SHRGY-GGGGS-SHRGY-GGGGGGGG-SHRGY-C | 59 |
| C-SHRGY-GGGGS-SHRGY-GGGGS-SHRGY-GGGGS-SHRGY-C | 60 |
| C-SHRGY-GGGGGGGG-SHRGY-PAPAP-SHRGY-GGGGGGGG-SHRGY-C | 61 |

In other embodiments, the cyclic synthetic peptide is prepared from a linear peptide by cyclization with sortase. “Cyclization with sortase” or “cyclized with sortase” refers to a method of cyclizing a linear peptide using the enzyme sortase. Sortase-based cyclization is known in the art for manufacturing large cyclic peptides. See, Bolscher, et al. (2011) FASEB J. 25(8):2650-2658, and references cited therein.

Butelase cyclization has also been used to cyclize peptides. Addition of the tripeptide Asn-His-Val motif at the C-terminus provides a substrate for butelase to cyclize a synthetic peptide at a rate significantly faster than that of sortase A. See, Nguyen, et al. (2016) Nat. Protocols 11:1977-88; Tam, et al. (June 2015) Peptides 2015: Proc. 24^{th} Am. Pept. Symp., Orlando, Fla., pg. 27.

One of skill in the art will recognize that the synthetic peptide of the invention will be beneficial for treating diseases. Accordingly, to facilitate administration, this invention also provides a composition containing one or more endogenous and/or synthetic peptides and a pharmaceutically acceptable carrier or excipient. The pharmaceutical compositions provided herein can be formulated for oral, ocular, intravenous, intravitreal, subconjunctival, subcutaneous, intramuscular, intraperitoneal, intracerebral, intraarterial, intraportal, intralesional, intrathecal, or intranasal administration or topical administration. Suitable pharmaceutical compositions can be determined by one skilled in the art depending upon, for example, the intended route of administration, delivery format and desired dosage. See, for example, Remington's Pharmaceutical Sciences (19th edition, 1995).

The synthetic peptide(s) can be incorporated in a conventional dosage form, such as a gel, wash, cream, tablet, capsule, pill, solution, eye drop, spray, bandage, contact lens, depot, injectable, implantable, or sustained-release formulation. The dosage forms may also include the necessary physiologically acceptable carrier material, excipient, lubricant, buffer, surfactant, antibacterial, bulking agent (such as mannitol), antioxidants (ascorbic acid or sodium bisulfite) or the like.

Acceptable formulation materials preferably are nontoxic to recipients at the dosages and concentrations employed. The pharmaceutical composition may contain formulation materials for modifying, maintaining or preserving, for example, the pH, osmolarity, viscosity, clarity, color, isotonicity, odor, sterility, stability, rate of dissolution or release, adsorption or penetration of the composition. Suitable formulation materials include, but are not limited to, amino acids (such as glycine, glutamine, asparagine, arginine or lysine); antimicrobials; antioxidants (such as ascorbic acid, sodium sulfite or sodium hydrogen-sulfite); buffers (such as borate, bicarbonate, Tris-HCl, citrates, phosphates or other organic acids); bulking agents (such as mannitol or glycine); chelating agents (such as ethylenediamine tetraacetic acid (EDTA)); complexing agents (such as caffeine, polyvinylpyrrolidone, beta-cyclodextrin or hydroxypropyl-beta-cyclodextrin); fillers; monosaccharides, disaccharides, and other carbohydrates (such as glucose, mannose or dextrins); proteins (such as serum albumin, gelatin or immunoglobulins); coloring, flavoring and diluting agents; emulsifying agents; hydrophilic polymers (such as polyvinylpyrrolidone); low molecular weight polypeptides; salt-forming counterions (such as sodium); preservatives (such as benzalkonium chloride, benzoic acid, salicylic acid, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents; surfactants or wetting agents (such as PLURONICS, PEG, sorbitan esters, polysorbates such as polysorbate 20 and polysorbate 80, TRITON, trimethamine, lecithin, cholesterol, or tyloxapal); stability enhancing agents (such as sucrose or sorbitol); tonicity enhancing agents (such as alkali metal halides, preferably sodium or potassium chloride, mannitol, or sorbitol); delivery vehicles; diluents; excipients and/or pharmaceutical adjuvants. See, for example, Remington's Pharmaceutical Sciences, Id.

The primary carrier or excipient in a pharmaceutical composition may be either aqueous or nonaqueous in nature. For example, a suitable carrier or excipient may be water for injection, physiological saline solution or artificial cerebrospinal fluid, possibly supplemented with other materials common in compositions for parenteral administration. Neutral buffered saline or saline mixed with serum albumin are further exemplary excipients. Pharmaceutical compositions can include Tris buffer of about pH 7.0-8.5, or acetate buffer of about pH 4.0-5.5, which may further include sorbitol or a suitable substitute. Pharmaceutical compositions of the invention may be prepared for storage by mixing the selected composition having the desired degree of purity with optional formulation agents (Remington's Pharmaceutical Sciences, Id.) in the form of a lyophilized cake or an aqueous solution. Further, the synthetic peptides of the invention may be formulated as a lyophilizate using appropriate excipients such as sucrose.

Administration routes for the pharmaceutical compositions of the invention include the oral route; injection by intravenous, intraperitoneal, intracerebral (intra-parenchymal), intracerebroventricular, intramuscular, intra-ocular, intraarterial, intraportal, or intralesional routes; or via sustained release systems or by implantation devices. The pharmaceutical compositions may be administered by bolus injection or continuously by infusion, or by implantation device. The pharmaceutical composition also can be administered locally via implantation of a membrane, sponge or another appropriate material onto which the synthetic histatin(s) has been absorbed or encapsulated. Where an implantation device is used, the device may be implanted into any suitable tissue or organ, and delivery of the endogenous or synthetic histatin(s) may be via diffusion, timed-release bolus, or continuous administration.

When parenteral administration is contemplated, the compositions for use in this invention may be in the form of a pyrogen-free, parenterally acceptable aqueous solution containing the endogenous or synthetic histatin(s) of the invention in a pharmaceutically acceptable vehicle. A particularly suitable vehicle for parenteral injection is sterile distilled water in which the synthetic peptide(s) is formulated as a sterile, isotonic solution, appropriately preserved. Preparation can involve the formulation of the synthetic peptide(s) with an agent, such as injectable microspheres, bio-erodible particles, polymeric compounds (such as polylactic acid or polyglycolic acid), beads or liposomes, that may provide controlled or sustained release of the synthetic peptide(s), which may then be delivered via a depot injection. In particular, formulation with hyaluronic acid has the effect of promoting sustained duration in the circulation.

The compositions may also be formulated for inhalation. In these embodiments, the synthetic peptide(s) of the invention is formulated as a dry powder for inhalation, or inhalation solutions may also be formulated with a propellant for aerosol delivery, such as by nebulization. Pulmonary administration is further described in, e.g., WO 1994/020069.

The pharmaceutical compositions of the invention can be delivered through the digestive tract, such as orally. The preparation of such pharmaceutically acceptable compositions is within the skill of the art. The synthetic peptide(s) of the invention that is administered in this fashion may be formulated with or without those carriers customarily used in the compounding of solid dosage forms such as tablets and capsules. A capsule may be designed to release the active portion of the formulation at the point in the gastrointestinal tract when bioavailability is maximized and pre-systemic degradation is minimized. Additional agents can be included to facilitate absorption of the synthetic peptide(s). Diluents, flavorings, low melting point waxes, vegetable oils, lubricants, suspending agents, tablet disintegrating agents, and binders may also be used.

These compositions may also contain adjuvants such as preservatives, wetting agents, emulsifying agents and dispersing agents. Prevention of the action of microorganisms can be ensured by the inclusion of various antibacterial and antifungal agents, for example, paraben, chlorobutanol, phenol sorbic acid and the like. It may also be desirable to include isotonic agents such as sugars, sodium chloride and the like. Prolonged absorption of an injectable pharmaceutical form can be brought about by the inclusion of agents which delay absorption such as aluminum monostearate and gelatin.

In certain embodiments, the synthetic peptide(s) is formulated for treating ocular diseases or conditions, both on the surface and inside the eye. In particular embodiments, the synthetic peptide(s) of the invention may be formulated and administered to the eye in drop form; topical gel form; as a solid formulation (e.g., similar to LACRISERT, hydroxypropyl cellulose ophthalmic insert); by injection into the anterior chamber of the eye; by injection into posterior chamber of the eye for inhibition of angiogenesis, inhibition of destructive MMP activity or to enhance epithelial wound healing; by coating of surgical devices (intraocular lens, glaucoma device, keratoprosthetic, lacrimal intubation tubes, lacrimal bypass tubes); by coating of contact lenses; or by coating of microbeads, nanobeads or other similar constructs.

As one skilled in the art will also appreciate, the composition described herein can be formulated so as to carry a minimum of adverse side effects. The compositions described herein can be suitable for long term use alone; useful as an adjunct therapy along with an antimicrobial agent (e.g., histatin, cystatin, lacritin, lactoferrin, LL-37), an antiviral agent, an antiparasitic agent, an immunomodulatory agent (e.g., glucocorticoids, cyclosporine, NSAIDs), an anti-scarring agent (e.g., mitomycin C or similar anti-metabolite), collagen, gelatin, a pain reliever, an anesthetic agent, or a combination thereof; and/or useful in a program involving rotation between any or all of these agents, thereby decreasing long term exposure to (and, therefore, side effects resulting from) any one agent.

This invention also provides kits containing one or more of the synthetic peptides, or a pharmaceutical composition containing the same, and optionally one or more of an antimicrobial agent, an antiviral agent, an antiparasitic agent, an immunomodulatory agent, an anti-scarring agent, collagen, gelatin, a pain reliever, or an anesthetic agent. Kits are typically provided in a suitable container (e.g., for example, a foil, plastic, or cardboard package). In certain embodiments, a kit may include one or more pharmaceutical excipients or carriers, pharmaceutical additives, and the like, as is described herein. In other embodiments, a kit may include a means for proper administration, such as, for example, graduated cups, syringes, needles, cleaning aids, an intraocular lens, a glaucoma device, an orbital implant, keratoprosthetic, lacrimal intubation tubes, lacrimal bypass tube, a contact lens and the like. In certain embodiments, a kit may include instructions for proper administration and/or preparation for proper administration.

Given the wound healing and epithelial cell migration promoting activity of the synthetic peptides disclosed herein, this invention also provides methods of promoting wound healing and/or epithelial cell migration by administering to a subject in need of such treatment one or more the synthetic peptides of this invention in an amount effective to promote wound healing and/or epithelial cell migration. “Subject,” as used herein, is meant to include humans, as well as non-human animals, particularly those who have a disease or condition, which may benefit from the promotion of wound healing and/or epithelial cell migration, e.g., as a therapy in the treatment of wounds or other body surface injuries connected to epithelial defects and relapsing epithelial erosion, such as surgical wounds, excision wounds, vesications, ulcers, other injuries, scratches, avulsive wounds, cuts, sordid wounds, furunculus and thermal or corrosive burns. Such wounds can be caused by both mechanical damage and other diseases, such as diabetes, corneal dystrophy, uremia, luck of nutrition, vitamin deficit, obesity, infection, immunodeficit or complications, connected with systematical use of steroids, radiotherapy, nonsteroidal anti-inflammatory drugs and anticancer drugs.

Notably, synthetic peptides including the SHRGY (SEQ ID NO:2) sequence have also been shown to increase ERK1/2 activation. Accordingly, the present invention also provides a method for increasing ERK activation by administering to a subject in need of such treatment one or more the synthetic peptides of this invention in an amount effective to increase ERK activation. It is well established that ERK modulation is important in both the innate and adaptive immune systems (Zhang & Dong (2005) Cell. Mol. Immunol. 2(1):20-27). As demonstrated herein, application of Hst5 increased signal intensity of phosphorylated ERK1/2 throughout the epithelial monolayer, indicating increased ERK1/2 activation of Hst5 to wounded epithelia. In addition, while untreated and SP1 peptide (which lacks SHRGY (SEQ ID NO:2)) treated samples had similar localization of pERK1/2, Hst5 and SP2 treated samples had elevated immunolocalization of pERK1/2 at the site of wound healing. This unexpected result indicates that the SHRGY (SEQ ID NO:2) peptide, which heretofore did not have any known ability to modulate ERK and would not have been predicted to do so based on functional domain understanding, can confer immunomodulatory activity to a synthetic peptide disclosed herein.

As used herein, the term “effective amount” or “therapeutically effective amount” refers to an amount of synthetic peptide of the invention or a pharmaceutical composition containing the synthetic peptide sufficient to achieve the stated desired result. In some aspects, an effective amount provides a measurable improvement in the rate epithelial cell migration, the rate or time to wound closure, and/or an increase in ERK and survival pathway modulation, as compared to a subject that has not received such treatment. The amount of the peptide which constitutes an “effective amount” or “therapeutically effective amount” may vary depending on the severity of the disease, the condition, weight, or age of the patient to be treated, the frequency of dosing, or the route of administration, but can be determined routinely by one of ordinary skill in the art. Depending on the location and condition to be treated, a dose in the range of 1 nanomolar to 500 micromolar or more of the synthetic peptide may be used. A clinician may titer the dosage or route of administration to obtain the optimal therapeutic effect. Typical dosages range from about 0.1 μg/kg to up to about 100 mg/kg or more, depending on the factors mentioned above. In certain embodiments, the dosage may range from 0.1 μg/kg up to about 100 mg/kg, or 1 μg/kg up to about 100 mg/kg, or 5 μg/kg up to about 100 mg/kg.

“Treating” a subject means accomplishing one or more of the following: (a) reducing the severity of the disease or condition; (b) arresting the development of the disease or condition; (c) inhibiting worsening of the disease or condition; (d) limiting or preventing recurrence of the d disease or condition in patients that have previously had the disease or condition; (e) causing regression of the disease or condition; (f) improving or eliminating the symptoms of the disease or condition; and/or (g) improving survival.

In accordance with this invention, synthetic peptides are of particular use in the treatment of ocular diseases or conditions including, but not limited to, ocular surface inflammatory disorders such as corneal inflammation (e.g., Mooren's or inflammatory and infectious ulcerations), necrotizing scleritis, ocular surface diseases mediated by inflammation, alkali burns, and chronic atopic diseases such as atopic or allergic conjunctivitis or eczematous diseases, fungal and bacterial infection, and corneal and conjunctival wounds, in particular wounds associated with neurotrophic/diabetic neuropathy.

In addition to the treatment of ocular diseases or conditions, the synthetic peptides can be tailored to promote wound healing and/or epithelial migration in other tissues or organs of interest, in particular in the treatment of wounds, inflammation, cancer, infection or injury. In one embodiment, lamellar tissues, nerve tissues, connective tissues, vascular tissues, muscle tissues, skeletal tissues, or blood components are treated. In another embodiment, organs such as skin, liver, lung, kidney, heart, or bowel are treated.

The following non-limiting examples are provided to further illustrate the present invention.

### EXAMPLE 1

### Materials and Methods

Peptide Synthesis. Histatin-5 peptides were synthesized using standard Fmoc based solid-phase synthesis chemistry on a Symphony Peptide Synthesizer (Protein Technologies, Tucson, Ariz.). The first amino acid (Fmoc-Tyr-OH) was covalently attached to the Wang resin. The peptide was synthesized in cycles, starting with the removal of Fmoc group in 20% piperidine in N,N-Dimethylformamide (DMF). The next amino acid was coupled using 0.1 M HBTU in DMF containing 0.4 M 4-methyl morpholine for 30 minutes ×2 and this process was continued to complete the synthesis. The resin-bound peptide was deprotected and cleaved from the resin using trifluoroacetic acid (TFA). Ethyl ether was added to precipitate the peptide from the TFA solution. The precipitated peptide was then dissolved in 50% acetonitrile in water and lyophilized. The crude peptide was purified on a Kinetex™ reversed-phase C18 column, 150×21.1 mm (Phenomenex, CA) using a BioCad SPRINT™ (Applied Biosystems, Foster City, Calif.) HPLC system. The pure peptide fraction was identified by electrospray ionization mass spectrometry (ESI MS) and lyophilized as appropriate. The cyclized version of SHRGY (SEQ ID NO:2) was prepared using a linker/spacer composed of 6-(Fmoc-amino)caproic acid, 6-(Fmoc-amino)hexanoic acid (C₂₁H₂₃NO₄).

The peptides were dissolved in cell culture grade water to obtain stock concentration of 10 mM and was stored at −20° C. Scrambled peptides (SP1, SP2, SP3, SP4 and SP5) were used as controls. SP1 was a 24 amino acid residue scrambled peptide based upon the full-length native Hst5 peptide. SP2 included 19 scrambled N-terminal amino acid residues (based upon the N-terminal 19 amino acid residues of native Hst5 peptide) and the SHRGY (SEQ ID NO:2) sequence at the C-terminus. SP3 and SP4 were scrambled versions of the SHRGY (SEQ ID NO:2) peptide and SP5 was a random pentapeptide with similar charge characteristics and molecular weight to SHRGY (SEQ ID NO:2). Table 5 shows the sequences of the peptides used in this study.

TABLE 5

| Name | Sequence | SEQ ID NO: |
|---|---|---|
| Histatin-5 | DSHAKRHHGYKRKFHEKHHSHRGY | 62 |
| (Hst5) | | |
| Hst5(1-]4) | DSHAKRHHGYKRKF | 63 |
| Hst5(1-19) | DSHAKRHHGYKRKFHEKHH | 64 |
| Hst5(1-21) | DSHAKRHIHGYKRKFHEKHHSH | 65 |
| Hst5(1-22) | DSHAKRHHGYKRKFHEKHHSHR | 66 |
| Hst5(1-23) | DSHAKRHHGYKRKFHEKHHSHRG | 67 |
| Hst5(5-24) | RHHGYKRKFHEKHHSHRGY | 68 |
| SHRGY | SHRGY | 2 |
| SP1 | YHGHRHFRKHKKHKEAHSYDRGSH | 69 |
| SP2 | HSHKEGHHYKRFKRKHHADSHRGY | 70 |
| SP3 | RYSGH | 71 |
| SP4 | RYHGS | 72 |
| SP5 | TNQQK | 73 |

Cell Culture. Human corneal limbal epithelial (HCLE) cells were cultured in keratinocyte-serum free medium (K-SFM; ThermoScientific, Waltham, Mass.) supplemented with 0.2 ng/mL rhEGF (ThermoScientific, Waltham, Mass.), bovine pituitary extract (ThermoScientific, Waltham, Mass.) and 1% Amphotericin B (ThermoScientific, Waltham, Mass.). Standard cell culture conditions (37° C., 5% CO₂, >95% humidity) were used during routine passages. The culture medium was replaced every 48 hours after seeding.

Human corneal epithelial (HCE) cells were cultured in a Medium Essential Media (MEM; Gibco, Life Technologies, Carlsbad, Calif.). HeLa cells were maintained in Dulbecco's Modified Eagle's Medium (DMEM; Life Technologies, Grand Island, N.Y.). Both media were supplemented with 10% fetal bovine serum (Gibco, Life Technologies, Carlsbad, Calif.) and 1% penicillin-streptomycin (Gibco, Life Technologies, Carlsbad, Calif.). The MCF-7 cell line was cultured in Roswell Park Memorial Institute 1640 (RPMI 1640; Gibco, Grand Island, N.Y.) supplemented with 10% fetal bovine serum (FBS; Gibco Life Technologies, Carlsbad, Calif.) and 1% penicillin-streptomycin (Gibco, Life Technologies, Carlsbad, Calif.) at 37° C. in a humidified atmosphere of 5% CO₂ and 95% air.

Short tandem repeat (STR) analysis was performed for each cell line to confirm authenticity of each cell line used.

In Vitro Scratch Assay. HCE, HCLE, HELA and MCF-7 cells were cultured in a 24-well plate at 2.5×10⁵ (cells/well) seeding density, and were grown to confluence on a 24-well plate. Subsequently, a straight-line scratch mark was made with a sterile P200 pipette tip. The cells were then washed twice with phosphate-buffered saline (PBS) to remove cellular debris. Wounded areas were then treated with the peptides provided in Table 5 at various concentrations in standard medium with reduced serum conditions (0.5% FBS in MEM medium (HCE), growth factor-free K-SFM media (HCLE), 1% FBS in DMEM medium (HeLa) and 0.5% FBS in RPMI 1640 medium (MCF-7)). Scratches were photographed microscopically at 4× magnification (Image Express Micro, Molecular Devices, San Jose, Calif.) every hour over the course of the experiment. The wound area at each time-point was measured using Image J software (Image J 1.47v, NIH, Thornwood, Bethesda, Md.). Relative wound closure was calculated by dividing the closure of the treated wound by that of the untreated wound. For all the experiments including truncated histatin-5, a final concentration of 80 μM was used. PD98059 (Calbiochem, San Diego, Calif.) at final concentration of 50 μM was used as a specific inhibitor of MEK. PD98059 was added to cell cultures at the same time as histatin peptides.

Wound Healing Assay. Corneal wounding experiments in mice were conducted in compliance with the Association for Research in Vision and Ophthalmology (ARVO) Statement for the Use of Animals in Ophthalmic and Vision Research. The protocol was approved by the Animal Care & Use Committee of the University of Illinois at Chicago. Twelve-to-nineteen week old C57BLl6J (Jackson Laboratory, Bar Harbor, Me.) mice were anesthetized with intraperitoneal injection of ketamine (100 mg/kg) and xylazine (5 mg/kg). After applying two drops of topical 0.5% proparacaine, a 2.0-mm area of the central epithelium was demarcated using 2-mm disposable biopsy punch and removed by an AlgerBrush II (The Alger Company, Lago Vista, Tex.). In the treatment (n=7) or the control (n=7) group, Histatin=5 (80 μM), SHRGY peptide (SEQ ID NO:2; 80 μM), or SP1 (80 μM) was applied to the cornea three times a day. At 0, 18 and 24 hours, the corneas were stained with fluorescein (FUL-GLO® Fluorescein Sodium ophthalmic strips, Akorn, Lake forest, Ill.) and photographed using a NIKON FS-2 photo-slit lamp with a NIKON D200 camera (Melville, N.Y.). Wound sizes were compared with the baseline for each mouse and the percentages of wound closure was measured using Image J software.

Cell Sprouting Assay. A cell sprouting assay was performed using HCE cells in a solubilized basement membrane sold under the tradename MATRIGEL® (reduced and diluted in MEM 1:1; Corning Life Sciences, Tewksbury, Mass.). HCE cells were embedded in the solubilized basement membrane at 5×10⁵ cells/10 μL spot. The cell spotted plates were then exposed to reduced serum media (0.5% FBS) (untreated negative control), Hst5 (50 μM), or 10% FBS (positive control). HCE cell migration was then followed with time lapse microscopy and imaged at 4× (Image Express Micro; Molecular Devices, CA). Cell covered area at a given time point was measured using Image J.

Western Blot. Western blot was performed following standard methods. Protein lysate (20 μg) was boiled in NuPAGE™ LDS sample buffer (Invitrogen, Carlsbad, Calif.) for 10 minutes and then subjected to electrophoresis on 12% NuPAGE™ Bis-Tris gels (Invitrogen, Carlsbad, Calif.), followed by transfer to nitrocellulose membranes (Amersham Protran, GE Healthcare, Pittsburgh, Pa.). Membranes were then blocked with Tris-buffered saline containing 3% nonfat dry milk for 1 hour and incubated with primary antibody against pERK1/2 (Cell Signaling, Danver, Mass.) (1:1000) overnight at 4° C. After washing in 0.05% Tris-buffered saline containing 0.05% polysorbate 20, membranes were then incubated for 1 hour with goat anti-rabbit-HRP (BD Biosciences, San Jose, Calif.) (1:2000) as the secondary antibody. The membranes were developed using X-ray film and ECL Pro solution (PerkinElmer, Waltham, Mass.). Beta-actin was used as an internal control.

Immunofluorescence Imaging. HCE cells were seeded in 8-well chamber slide at 9×10⁴ (cells/well) seeding density, and allowed to incubate to form a confluent monolayer. Subsequently, a straight-line scratch mark was made with a sterile P10 pipette tip. The cells were then washed with media to remove cellular debris. Wounded areas were subsequently left untreated or treated with Hst5, SP1 or SP2 at 80 μM concentrations in MEM media with 0.5% FBS for 45 minutes. Cells were then fixed with 4% paraformaldehyde for 30 minutes and permeabilized with 0.1% Triton X-100 for 5 minutes. After washing with PBS, cells were incubated at room temperature for 30 minutes with 5% bovine serum albumin (BSA) and 5% normal goat serum in PBS. Cells were subsequently incubated with primary antibodies diluted in 1% BSA against p-ERK1/2 (1:200) (Cell Signaling, Danver, Mass.) at 4° C. for 16 hours. After three washings with PBS, cells were incubated with fluorescein isothiocyanate-conjugated sheep anti-rabbit IgG antibody (BD Biosciences, San Jose, Calif.) (1:500) diluted in 1% BSA at room temperature for 60 minutes. After extensive washing with PBS, the cells were then stained with 4′,6-diamidino-2-phenylindole (DAPI; (Roche, Mannheim, GE) for 2 minutes for nuclear staining. The cells were mounted in Fluoro gel with Tris buffer (Electron Microscopy Sciences, Hattfield, Pa.) and observed under a confocal microscope (Zeiss LSM 710 Confocal Microscope, Oberkochen, Germany) using a 10× objective.

For immunofluorescence, excised whole mouse eye was snap frozen in optimal cutting temperature compound (OCT; (Fisher Healthcare, Galderma, Calif.). The frozen tissues were cut into 10 μm cryosections (ThermoScientific NX50 Cryomicrotome, Waltham, Mass.) and subsequently were mounted on Superfrost plus slides (Thermofisher, Waltham, Mass.). Slides were fixed for 20 minutes in methanol, washed several times with PBS, stained with DAPI for 2 minutes, and further washed with PBS and deionized water. The slides were then mounted in Fluoro gel with Tris buffer (Electron Microscopy Sciences, Hattfield, Pa.) and observed under a confocal microscope (Zeiss LSM 710 Confocal Microscope, Oberkochen, Germany) using a 10× objective.

Statistical Analysis. Experiments were analyzed using two way or one-way ANOYA followed by Bonferroni's or Dunnett's post hoc tests or the Student's t-test as appropriate. p values <0.05 were considered statistically significant. Statistical analyses were performed using GraphPad Prism software 7.0 (GraphPad Software, La Jolla, Calif.).

A power analysis to determine the sample size for the murine corneal wounding experiment was performed using G-Power to compare between treatment (SHRGY (SEQ ID NO:2) or Hst5) and control (SP1) groups. Parameters for calculation included a Beta of 0.8, an Alpha of 0.05, and an effect size of 25% and yielded a per group sample size of n=6.

### EXAMPLE 2

### Histatin-5 Promotes Cell Migration

A cell sprouting assay using HCE cells embedded as a spot in growth factor reduced solubilized basement membrane sold under the tradename MATRIGEL® (Corning Life Sciences, Tewksbury, Mass.) was used to determine if HST5 could promote epithelial cell migration. At 72 hours, there was a statistically significant increase in cell migration in Hst5 (50 μM) treated condition compared to vehicle only control. A standardized scratch assay using the HCLE cell line tested the effects of Hst5 on cell migration. HCLE cells were grown to confluence and mechanically scratched using a pipette tip. Cells were treated with different concentrations (20, 50, 80 and 100 μM) of Hst5 or left untreated as a control. Time-lapse microscopy was performed and wound areas were analyzed at different time points. This analysis demonstrated a dose-dependent increase in rates of in vitro scratch assay closure with application of Hst5 compared with untreated control. The most significant increase in scratch closure rates was noticed at 50 μM. These findings were corroborated in the HCE corneal cell line with a statistically significant peak effect at 80 μM as compared with scrambled peptide control (SP1). Statistically significant effects were also observed in the HeLa cell line and MCF-7 breast cancer cell line.

### EXAMPLE 3

### C-Terminal SHR Domain of Hst5 is Required for Promoting Epithelial Cell Migration

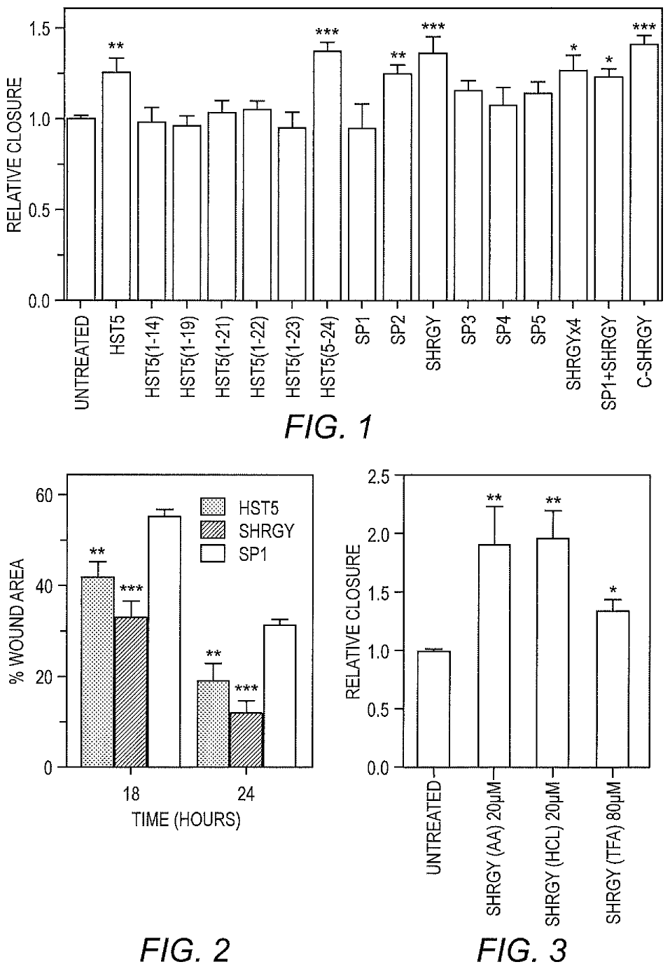

To identify the residues of Hst5 required for epithelial cell migration, serial truncation experiments were performed, progressively deleting residues in Hst5. This analysis indicated that the C-terminal SHRGY (SEQ ID NO:2) residues of Hst5 were necessary and sufficient to promote migration (FIG. 1). All peptides were tested at 80 μM. Truncated versions of Hst5, which did not include the C-terminal SHRGY (SEQ ID NO:2) sequence, i.e., Hst5(1-14), Hst5(1-19), Hst5(1-21), Hst5(1-22) and Hst5(1-23), showed no significant increase in wound closure rates (FIG. 1). However, constructs containing an intact SHRGY sequence, i.e., Hst5, Hst5(5-24), SP2, SHRGY (SEQ ID NO:2), a multimer of SHRGY (SEQ ID NO:2) composed of 4 repeating units of said sequence (i.e., SHRGY-(CH₂)₆-SHRGY-(CH₂)₆-SHRGY-(CH₂)₆-SHRGY (SEQ ID NO:51) and a cyclized SHRGY (SEQ ID NO:2) peptide (c-SHRGY) showed significant increases in scratch closure rates (FIG. 1). Scrambled peptides SP3 and SP4 did not significantly increase wound closure rates. Likewise, the random pentapeptide SP5 with similar molecular weight and charge characteristics to SHRGY (SEQ ID NO:2) showed no significant improvement in the wound closure rates. Thus, the SHRGY (SEQ ID NO:2) sequence is necessary and sufficient to promote epithelial migration rates in in vitro scratch assays.

### EXAMPLE 4

### ERK Activation is Necessary for the Pro-Migratory Effects of Hst5

The level of activation/phosphorylation of ERK with and without wounding and with and without Hst5 application were investigated to determine whether the cellular signaling pathways underpinning the pro-migratory effects of Hst5 were similar to those of Hst1 in other epithelial cell types. Wounding causes an increased in phosphorylated form of ERK1/2 (p-ERK1/2). Immunolocalization was used to determine whether Hst5 application to a scratched sheet of epithelium would affect pERK1/2 levels. Western blot analysis confirmed that wounding increased pERK1/2 levels alone, and that these levels were further increased by Hst5 application. The application of the SHRGY (SEQ ID NO:2)-containing scrambled peptide SP2 increased pERK1/2 levels to a comparable intensity to Hst5. SP1, which does not contain SHRGY (SEQ ID NO:2), did not elicit the same increase in pERK1/2 immunolocalization as did the SHRGY (SEQ ID NO:2)-containing peptides. Co-treatment of Hst5 and MEK specific inhibitor PD98059 eliminated the effects of Hst5 on promoting wound closure indicating that Hst5 effects require ERK activation.

### EXAMPLE 5

### Hst5 Application Promotes Wound Healing in a Murine Corneal Injury Model

Using a standard mouse model of corneal injury, it was observed that topical administration of Hst5 or SHRGY (SEQ ID NO:2) peptide, provided a significant improvement in corneal wound closure rates at a level which was superior to scrambled peptide control (SP1) (FIG. 2). Histologic analysis of the wounded corneas (DAPI staining on the cross sections of the cornea) demonstrated pathologic evidence of reductions in corneal wound size in the Hst5-treated condition compared to SP1-treated controls. Thus, Hst5 and SHRGY (SEQ ID NO:2)-containing peptides can enhance wound healing in a well-vetted model of murine corneal epithelial injury.

### EXAMPLE 6

### Toxicity and Salt Forms

Human corneal epithelial cells exposed to increasing concentrations of the SHRGY (SEQ ID NO:2) pentapeptide, i.e., 31.25 μM, 62.5 μM, 125 μM, 250 μM, 500 μM, 1000 μM, 2000 μM, 4000 μM and 8000 μM, indicated a minimal reduction in cell viability at concentrations at or less than 4000 μM after 24 hours, as determined using a conventional WST1 assay, or induced cell death up to above 4000 μM concentrations at 24 hours in an LDH assay.

Cell toxicity of different salt forms of the SHRGY (SEQ ID NO:2) pentapeptide was also examined. Three different salt forms of SHRGY (SEQ ID NO:2), i.e., acetic acid, hydrochloride and trifluoroacetate were prepared and toxicity, as determined using a conventional WST1 assay, was analyzed at 15.625 μM, 31.25 μM, 62.5 μM, 125 μM, 250 μM and 500 μM. All three salt forms of SHRGY (SEQ ID NO:2) showed no significant toxicity compared to untreated sample at any of the peptide concentrations tested.

Wound closure rates of the three salt forms were also analyzed in accordance with the methods disclosed herein. In particular, human corneal epithelial cells were scratched using standardized scratch assays and rates of wound healing were measured after application of the acetic acid, hydrochloride and trifluoroacetate salt forms of SHRGY (SEQ ID NO:2). All tested forms exhibited enhanced rates of wound closure (FIG. 3).

What is claimed is:

1. A synthetic peptide, or pharmaceutically acceptable salt thereof, comprising the structure of Formula I:
Z—R¹-[L-R²]ₙ (I)
wherein
at least one of R¹ or R² is a 5 to 10 amino acid residue peptide comprising the amino acid sequence SHXGY (SEQ ID NO:1), wherein X is R, K, H, D or E and the other of R¹ or R² is a metal binding peptide, wound healing peptide, or antimicrobial peptide;
Z is present or absent and when present is an exogenous peptide;
L is a linker; and
n is 0 or ≥1
with the proviso that when n is 0, R¹ is a 5 to 10 amino acid residue peptide comprising the amino acid sequence SHXGY (SEQ ID NO:1).

2. The synthetic peptide of claim 1, wherein the linker is a hydrocarbon linker.

3. The synthetic peptide of claim 1, wherein each L may be the same or different linker.

4. The synthetic peptide of claim 1, wherein said synthetic peptide is linear or cyclized.

5. The synthetic peptide of claim 1, wherein said synthetic peptide comprises a modification selected from glycosylation, acetylation, amidation, formylation, hydroxylation, methylation, myristoylation, phosphorylation, sulfonation, PEGylation or lipidation.

6. The synthetic peptide of claim 1, wherein the metal binding peptide comprises the amino acid sequence HEXXH (SEQ ID NO:14), wherein X is K, R, or H.

7. The synthetic peptide of claim 1, wherein the wound healing peptide comprises the amino acid sequence SNYLYDN (SEQ ID NO:26) or SHXGY (SEQ ID NO:1), wherein X is R, K, H, D or E.

8. The synthetic peptide of claim 1, wherein the antimicrobial peptide comprises the amino acid sequence RKFHEKHHSHRGYR (SEQ ID NO:28) or AKRHHGYKRKFH (SEQ ID NO:29).

9. A pharmaceutical composition comprising one or more synthetic peptides, or pharmaceutically acceptable salts thereof, of claim 1 and a pharmaceutically acceptable carrier or excipient.

10. The pharmaceutical composition of claim 9, wherein said pharmaceutical composition is formulated for topical, oral, ocular, intravenous, intravitreal, subconjunctival, subcutaneous, intramuscular, intraperitoneal, intracerebral, intraarterial, intraportal, intralesional, intrathecal, or intranasal administration.

11. The pharmaceutical composition of claim 9, wherein said pharmaceutical composition is in the form of a gel, wash, cream, tablet, capsule, pill, solution, eye drop, spray, bandage, contact lens, depot, injectable, implantable, or sustained-release formulation.

12. A method for promoting wound healing or epithelial cell migration comprising administering to a subject in need of treatment an effective amount of a synthetic peptide, or pharmaceutically acceptable salt thereof, of claim 1 thereby promoting wound healing or epithelial cell migration.

13. The method of claim 12, wherein the amount of synthetic peptide, or pharmaceutically acceptable salt thereof, is in the range of 1 nanomolar to 500 micromolar.

14. The method of claim 12, further comprising administering an antimicrobial agent, an antiviral agent, an antiparasitic agent, an anti-scarring agent, an immunomodulatory agent, collagen, gelatin, a pain reliever, an anesthetic agent, or a combination thereof.

15. A method for promoting wound healing or epithelial cell migration comprising administering to a subject in need of treatment an effective amount of a pharmaceutical composition of claim 9 thereby promoting wound healing or epithelial cell migration.

16. The method of claim 15, wherein the amount of synthetic peptide, or pharmaceutically acceptable salt thereof, is in the range of 1 nanomolar to 500 micromolar.

17. The method of claim 15, further comprising administering an antimicrobial agent, an antiviral agent, an antiparasitic agent, an anti-scarring agent, an immunomodulatory agent, collagen, gelatin, a pain reliever, an anesthetic agent, or a combination thereof.

18. A method for increasing extracellular signal-regulated protein kinase (ERK) activation comprising administering to a subject in need of treatment an effective amount of a synthetic peptide, or pharmaceutically acceptable salt thereof, of claim 1 thereby increasing ERK activation.

19. A kit comprising one or more synthetic peptides, or pharmaceutically acceptable salts thereof, of claim 1.

20. The kit of claim 19, further comprising an antimicrobial agent, an antiviral agent, an antiparasitic agent, an anti-fungal agent, an anti-scarring agent, an immunomodulatory agent, collagen, gelatin, a pain reliever, an anesthetic agent, or a combination thereof.


SEQUENCE LISTING (73 sequences)

SEQ ID NO: 1 - protein, 5 aa, Artificial Sequence
  Synthetic peptide
  MISC_FEATURE (3)..(3): X denotes Arg, Lys, His, Asp or Glu
SHXGY

SEQ ID NO: 2 - protein, 5 aa, Artificial Sequence
  Synthetic peptide
SHRGY

SEQ ID NO: 3 - protein, 5 aa, Artificial Sequence
  Synthetic peptide
SHDGY

SEQ ID NO: 4 - protein, 5 aa, Artificial Sequence
  Synthetic peptide
SHKGY

SEQ ID NO: 5 - protein, 5 aa, Artificial Sequence
  Synthetic peptide
SHHGY

SEQ ID NO: 6 - protein, 5 aa, Artificial Sequence
  Synthetic peptide
SHEGY

SEQ ID NO: 7 - protein, 32 aa, Artificial Sequence
  Synthetic peptide
DSHAKRHHGYKRKFHEKHHSHRGYRSNYLYDN

SEQ ID NO: 8 - protein, 10 aa, Artificial Sequence
  Synthetic peptide
HHSHRGYRSN

SEQ ID NO: 9 - protein, 10 aa, Artificial Sequence
  Synthetic peptide
HEKHHSHRGY

SEQ ID NO: 10 - protein, 10 aa, Artificial Sequence
  Synthetic peptide
EKHHSHRGYR

SEQ ID NO: 11 - protein, 8 aa, Artificial Sequence
  Synthetic peptide
KHHSHRGY

SEQ ID NO: 12 - protein, 7 aa, Artificial Sequence
  Synthetic peptide
HHSHRGY

SEQ ID NO: 13 - protein, 6 aa, Artificial Sequence
  Synthetic peptide
HSHRGY

SEQ ID NO: 14 - protein, 5 aa, Artificial Sequence
  Synthetic peptide
  MISC_FEATURE (3)..(4): X denotes Lys, Arg, or His
HEXXH

SEQ ID NO: 15 - protein, 5 aa, Artificial Sequence
  Synthetic peptide
HEKKH

SEQ ID NO: 16 - protein, 5 aa, Artificial Sequence
  Synthetic peptide
HEKRH

SEQ ID NO: 17 - protein, 5 aa, Artificial Sequence
  Synthetic peptide
HEKHH

SEQ ID NO: 18 - protein, 5 aa, Artificial Sequence
  Synthetic peptide
HERKH

SEQ ID NO: 19 - protein, 5 aa, Artificial Sequence
  Synthetic peptide
HERRH

SEQ ID NO: 20 - protein, 5 aa, Artificial Sequence
  Synthetic peptide
HERHH

SEQ ID NO: 21 - protein, 5 aa, Artificial Sequence
  Synthetic peptide
HEHKH

SEQ ID NO: 22 - protein, 5 aa, Artificial Sequence
  Synthetic peptide
HEHRH

SEQ ID NO: 23 - protein, 5 aa, Artificial Sequence
  Synthetic peptide
HEHHH

SEQ ID NO: 24 - protein, 14 aa, Artificial Sequence
  Synthetic peptide
GYKRKFHEKHHSHR

SEQ ID NO: 25 - protein, 6 aa, Artificial Sequence
  Synthetic peptide
HEKRHH

SEQ ID NO: 26 - protein, 7 aa, Artificial Sequence
  Synthetic peptide
SNYLYDN

SEQ ID NO: 27 - protein, 12 aa, Artificial Sequence
  Synthetic peptide
YGDYGSNYLYDN

SEQ ID NO: 28 - protein, 14 aa, Artificial Sequence
  Synthetic peptide
RKFHEKHHSHRGYR

SEQ ID NO: 29 - protein, 12 aa, Artificial Sequence
  Synthetic peptide
AKRHHGYKRKFH

SEQ ID NO: 30 - protein, 25 aa, Artificial Sequence
  Synthetic peptide
  MISC_FEATURE (6)..(9): Xaa is present or absent and when present denotes Gly
  MISC_FEATURE (10)..(10): Xaa is present or absent and when present denotes Ser
  MISC_FEATURE (11)..(14): Xaa is present or absent and when present denotes Gly
  MISC_FEATURE (15)..(15): Xaa is present or absent and when present denotes Ser
  MISC_FEATURE (16)..(19): Xaa is present or absent and when present denotes Gly
  MISC_FEATURE (20)..(20): Xaa is present or absent and when present denotes Ser
  MISC_FEATURE (21)..(24): Xaa is present or absent and when present denotes Gly
  MISC_FEATURE (25)..(25): Xaa is present or absent and when present denotes Ser
GGGGSXXXXXXXXXXXXXXXXXXXX

SEQ ID NO: 31 - protein, 18 aa, Artificial Sequence
  Synthetic peptide
KESGSVSSEQLAQFRSLD

SEQ ID NO: 32 - protein, 14 aa, Artificial Sequence
  Synthetic peptide
EGKSSGSGSESKST

SEQ ID NO: 33 - protein, 8 aa, Artificial Sequence
  Synthetic peptide
GGGGGGGG

SEQ ID NO: 34 - protein, 12 aa, Artificial Sequence
  Synthetic peptide
GSAGSAAGSGEF

SEQ ID NO: 35 - protein, 20 aa, Artificial Sequence
  Synthetic peptide
  MISC_FEATURE (5)..(6): Xaa is present or absent and when present denotes Gly
  MISC_FEATURE (7)..(7): Xaa is present or absent and when present denotes Ser
  MISC_FEATURE (8)..(10): Xaa is present or absent and when present denotes Gly
  MISC_FEATURE (11)..(11): Xaa is present or absent and when present denotes Ser
  MISC_FEATURE (12)..(14): Xaa is present or absent and when present denotes Gly
  MISC_FEATURE (15)..(15): Xaa is present or absent and when present denotes Ser
  MISC_FEATURE (16)..(18): Xaa is present or absent and when present denotes Gly
  MISC_FEATURE (19)..(19): Xaa is present or absent and when present Ser
  MISC_FEATURE (20)..(20): Xaa is present or absent and when present Gly
GGSGXXXXXXXXXXXXXXXX

SEQ ID NO: 36 - protein, 10 aa, Artificial Sequence
  Synthetic peptide
  MISC_FEATURE (3)..(3): Xaa is present or absent and when present denotes Gly
  MISC_FEATURE (4)..(4): Xaa is present or absent and when present denotes Ser
  MISC_FEATURE (5)..(5): Xaa is present or absent and when present denotes Gly
  MISC_FEATURE (6)..(6): Xaa is present or absent and when present denotes Ser
  MISC_FEATURE (7)..(7): Xaa is present or absent and when present denotes Gly
  MISC_FEATURE (8)..(8): Xaa is present or absent and when present denotes Ser
  MISC_FEATURE (9)..(9): Xaa is present or absent and when present denotes Gly
  MISC_FEATURE (10)..(10): Xaa is present or absent and when present denotes Ser
GSXXXXXXXX

SEQ ID NO: 37 - protein, 25 aa, Artificial Sequence
  Synthetic peptide
  MISC_FEATURE (6)..(6): Xaa is present or absent and when present denotes Glu
  MISC_FEATURE (7)..(9): Xaa is present or absent and when present denotes Ala
  MISC_FEATURE (10)..(10): Xaa is present or absent and when present denotes Lys
  MISC_FEATURE (11)..(11): Xaa is present or absent and when present denotes Glu
  MISC_FEATURE (12)..(14): Xaa is present or absent and when present denotes Ala
  MISC_FEATURE (15)..(15): Xaa is present or absent and when present denotes Lys
  MISC_FEATURE (16)..(16): Xaa is present or absent and when present denotes Glu
  MISC_FEATURE (17)..(19): Xaa is present or absent and when present denotes Ala
  MISC_FEATURE (20)..(20): Xaa is present or absent and when present denotes Lys
  MISC_FEATURE (21)..(21): Xaa is present or absent and when present denotes Glu
  MISC_FEATURE (22)..(24): Xaa is present or absent and when present denotes Ala
  MISC_FEATURE (25)..(25): Xaa is present or absent and when present denotes Lys
EAAAKXXXXXXXXXXXXXXXXXXXX

SEQ ID NO: 38 - protein, 27 aa, Artificial Sequence
  Synthetic peptide
  MISC_FEATURE (7)..(7): Xaa is present or absent and when present denotes Glu
  MISC_FEATURE (8)..(10): Xaa is present or absent and when present denotes Ala
  MISC_FEATURE (11)..(11): Xaa is present or absent and when present denotes Lys
  MISC_FEATURE (12)..(12): Xaa is present or absent and when present denotes Glu
  MISC_FEATURE (13)..(15): Xaa is present or absent and when present denotes Ala
  MISC_FEATURE (16)..(16): Xaa is present or absent and when present denotes Lys
  MISC_FEATURE (17)..(17): Xaa is present or absent and when present denotes Glu
  MISC_FEATURE (18)..(20): Xaa is present or absent and when present denotes Ala
  MISC_FEATURE (21)..(21): Xaa is present or absent and when present denotes Lys
  MISC_FEATURE (22)..(22): Xaa is present or absent and when present denotes Glu
  MISC_FEATURE (23)..(25): Xaa is present or absent and when present denotes Ala
  MISC_FEATURE (26)..(26): Xaa is present or absent and when present denotes Lys
AEAAAKXXXXXXXXXXXXXXXXXXXXA

SEQ ID NO: 39 - protein, 5 aa, Artificial Sequence
  Synthetic peptide
PAPAP

SEQ ID NO: 40 - protein, 10 aa, Artificial Sequence
  Synthetic peptide
  MISC_FEATURE (1)..(1): Xaa denotes any amino acid residue
  MISC_FEATURE (3)..(3): Xaa is present or absent and when present denotes any amino acid residue
  MISC_FEATURE (4)..(4): Xaa is present or absent and when present denotes Pro
  MISC_FEATURE (5)..(5): Xaa is present or absent and when present denotes any amino acid residue
  MISC_FEATURE (6)..(6): Xaa is present or absent and when present denotes Pro
  MISC_FEATURE (7)..(7): Xaa is present or absent and when present denotes any amino acid residue
  MISC_FEATURE (8)..(8): Xaa is present or absent and when present denotes Pro
  MISC_FEATURE (9)..(9): Xaa is present or absent and when present denotes any amino acid residue
  MISC_FEATURE (10)..(10): Xaa is present or absent and when present denotes Pro
XPXXXXXXXX

SEQ ID NO: 41 - protein, 17 aa, Artificial Sequence
  Synthetic peptide
VSQTSKLTRAETVFPDV

SEQ ID NO: 42 - protein, 6 aa, Artificial Sequence
  Synthetic peptide
PLGLWA

SEQ ID NO: 43 - protein, 6 aa, Artificial Sequence
  Synthetic peptide
RVLAEA

SEQ ID NO: 44 - protein, 10 aa, Artificial Sequence
  Synthetic peptide
EDVVCCSMSY

SEQ ID NO: 45 - protein, 8 aa, Artificial Sequence
  Synthetic peptide
GGIEGRGS

SEQ ID NO: 46 - protein, 10 aa, Artificial Sequence
  Synthetic peptide
TRHRQPRGWE

SEQ ID NO: 47 - protein, 10 aa, Artificial Sequence
  Synthetic peptide
AGNRVRRSVG

SEQ ID NO: 48 - protein, 9 aa, Artificial Sequence
  Synthetic peptide
RRRRRRRRR

SEQ ID NO: 49 - protein, 4 aa, Artificial Sequence
  Synthetic peptide
GFLG

SEQ ID NO: 50 - protein, 12 aa, Artificial Sequence
  Synthetic peptide
CRRRRRREAEAC

SEQ ID NO: 51 - protein, 20 aa, Artificial Sequence
  Synthetic peptide
  NON_CONS (5)..(6): Residues linked by -(CH2)6- hydrocarbon
  NON_CONS (10)..(11): Residues linked by -(CH2)6- hydrocarbon
  NON_CONS (15)..(16): Residues linked by -(CH2)6- hydrocarbon
SHRGYSHRGYSHRGYSHRGY

SEQ ID NO: 52 - protein, 20 aa, Artificial Sequence
  Synthetic peptide
  NON_CONS (5)..(6): Residues linked by -(CH2)6- hydrocarbon
  NON_CONS (10)..(11): Residues linked by -(CH2)6- hydrocarbon
  NON_CONS (15)..(16): Residues linked by -(CH2)6- hydrocarbon
SHRGYSHDGYSHRGYSHDGY

SEQ ID NO: 53 - protein, 41 aa, Artificial Sequence
  Synthetic peptide
SHRGYGGGGGGGGSHRGYGGGGSSHRGYGGGGGGGGSHRGY

SEQ ID NO: 54 - protein, 35 aa, Artificial Sequence
  Synthetic peptide
SHRGYGGGGSSHRGYGGGGSSHRGYGGGGSSHRGY

SEQ ID NO: 55 - protein, 41 aa, Artificial Sequence
  Synthetic peptide
SHRGYGGGGGGGGSHRGYPAPAPSHRGYGGGGGGGGSHRGY

SEQ ID NO: 56 - protein, 16 aa, Artificial Sequence
  Synthetic peptide
RQIKIWFQNRRMKWKK

SEQ ID NO: 57 - protein, 22 aa, Artificial Sequence
  Synthetic peptide
  NON_CONS (6)..(7): Residues linked by -(CH2)6- hydrocarbon
  NON_CONS (11)..(12): Residues linked by -(CH2)6- hydrocarbon
  NON_CONS (16)..(17): Residues linked by -(CH2)6- hydrocarbon
CSHRGYSHRGYSHRGYSHRGYC

SEQ ID NO: 58 - protein, 22 aa, Artificial Sequence
  Synthetic peptide
  NON_CONS (6)..(7): Residues linked by -(CH2)6- hydrocarbon
  NON_CONS (11)..(12): Residues linked by -(CH2)6- hydrocarbon
  NON_CONS (16)..(17): Residues linked by -(CH2)6- hydrocarbon
CSHRGYSHDGYSHRGYSHDGYC

SEQ ID NO: 59 - protein, 43 aa, Artificial Sequence
  Synthetic peptide
CSHRGYGGGGGGGGSHRGYGGGGSSHRGYGGGGGGGGSHRGYC

SEQ ID NO: 60 - protein, 37 aa, Artificial Sequence
  Synthetic peptide
CSHRGYGGGGSSHRGYGGGGSSHRGYGGGGSSHRGYC

SEQ ID NO: 61 - protein, 43 aa, Artificial Sequence
  Synthetic peptide
CSHRGYGGGGGGGGSHRGYPAPAPSHRGYGGGGGGGGSHRGYC

SEQ ID NO: 62 - protein, 24 aa, Artificial Sequence
  Synthetic peptide
DSHAKRHHGYKRKFHEKHHSHRGY

SEQ ID NO: 63 - protein, 14 aa, Artificial Sequence
  Synthetic peptide
DSHAKRHHGYKRKF

SEQ ID NO: 64 - protein, 19 aa, Artificial Sequence
  Synthetic peptide
DSHAKRHHGYKRKFHEKHH

SEQ ID NO: 65 - protein, 21 aa, Artificial Sequence
  Synthetic peptide
DSHAKRHHGYKRKFHEKHHSH

SEQ ID NO: 66 - protein, 22 aa, Artificial Sequence
  Synthetic peptide
DSHAKRHHGYKRKFHEKHHSHR

SEQ ID NO: 67 - protein, 23 aa, Artificial Sequence
  Synthetic peptide
DSHAKRHHGYKRKFHEKHHSHRG

SEQ ID NO: 68 - protein, 19 aa, Artificial Sequence
  Synthetic peptide
RHHGYKRKFHEKHHSHRGY

SEQ ID NO: 69 - protein, 24 aa, Artificial Sequence
  Synthetic peptide
YHGHRHFRKHKKHKEAHSYDRGSH

SEQ ID NO: 70 - protein, 24 aa, Artificial Sequence
  Synthetic peptide
HSHKEGHHYKRFKRKHHADSHRGY

SEQ ID NO: 71 - protein, 5 aa, Artificial Sequence
  Synthetic peptide
RYSGH

SEQ ID NO: 72 - protein, 5 aa, Artificial sequence
  Synthetic peptide
RYHGS

SEQ ID NO: 73 - protein, 5 aa, Artificial sequence
  Synthetic peptide
TNQQK